(19) United States
(12) Patent Application Publication
(10) Pub. No.: US 2019/0365244 A1
(43) Pub. Date: Dec. 5, 2019

(54) NON-CONTACT PULSE TRANSIT TIME MEASUREMENT SYSTEM AND NON-CONTACT VITAL SIGN SENSING DEVICE THEREOF

(71) Applicant: NATIONAL SUN YAT-SEN UNIVERSITY, Kaohsiung City (TW)

(72) Inventors: Tzyy-Sheng Horng, Kaohsiung City (TW); Fu-Kang Wang, Kaohsiung City (TW); Mu-Cyun Tang, Kaohsiung City (TW); Chien-Min Liao, Kaohsiung City (TW)

(21) Appl. No.: 16/379,937

(22) Filed: Apr. 10, 2019

(30) Foreign Application Priority Data
May 31, 2018 (TW) 107118813

(51) Int. Cl. A61B 5/0205; G01S 13/88; G01S 7/35

(52) U.S. Cl. CPC A61B 5/0205; A61B 5/0816; G01S 7/35; G01S 13/88

(57) ABSTRACT

In non-contact pulse transit time measurement system of the present invention, two continuous-wave radars are provided to detect movements at two positions on a subject for use in measuring pulse transit time. The measurement of the pulse transit time can be continuous and last for a long time because there is no contact to skin necessary.

### FIELD OF THE INVENTION

This invention generally relates to a pulse transit time measurement system, and more particularly to a non-contact pulse transit time measurement system.

### BACKGROUND OF THE INVENTION

Pulse transit time (PTT) is the time required for the pulse wave passing through an appropriate length of artery to calculate the pulse wave velocity that can be used to estimate the blood pressure (BP). Compared to the conventional cuff-based BP measurement, the PTT-based BP measurement can be continuous for as long as is needed because it is cuff-less.

FIG. 1 shows the commercial PTT measurement system depending on chest ECG (Electrocardiography) and finger PPG (Photoplethysmography), and the obtained ECG and PPG signals are delivered to a bio-system BS to analyze the PTT. However, multiple electrodes must be placed on the skin of subject's chest or limbs for ECG measurement, and an infrared sensor has to be clipped on subject's finger for PPG measurement. Both ECG and PPG devices require direct skin contact and the subject may feel uncomfortable or painful during a long-term measurement period so that the commercial PTT measurement system is not favorable for continuous BP monitoring.

Doppler radars have been extensively used to monitor health by detecting tiny body movements due to vital signs such as respiration and pulse. A patent publication US 2014/0171811 discloses a vital sign sensing system that utilizes two expensive ultra-wideband (UWB) impulse radars to measure the PTT between two positions on a human body. The penetrating capability of UWB signals is insufficient because the transmit power is severely limited by regulation. Therefore, the antennas in the system must be placed close to human skin for detecting pulse wave signals, which makes the distance between two measurement positions too short (less than 10 cm) to accurately calculate the pulse wave velocity from the PTT for BP estimation.

### SUMMARY

The object of the present invention is to detect movements at two positions on a subject by using two continuous-wave (CW) radars without contact and then extract pulse transit time (PTT) from the movement waveforms measured at the two positions.

One aspect of the present invention provides a system for non-contact PTT measurement. The non-contact PTT measurement system includes a non-contact vital sign sensing device and a computer. The non-contact vital sign sensing device includes a first CW radar and a second CW radar. The first CW radar configured to transmit a first wireless signal to a first position on a subject, receive a first reflected signal reflected from the first position and perform demodulation according to the first reflected signal to obtain a first demodulated signal. The second CW radar configured to transmit a second wireless signal to a second position on the subject, receive a second reflected signal reflected from the second position and perform demodulation according to the second reflected signal to obtain a second demodulated signal. The computer coupled to the first and second CW radars of the non-contact vital sign sensing device for receiving the first and second demodulated signals from the first and second CW radars and configured to extract a PTT from the first and second demodulated signals.

The first and second CW radars in the present invention are provided to detect the movements at the first and second position on the subject, respectively, for measuring the PTT between the two positions. The first and second CW radars are both non-contact devices so continuous PTT measurement can be performed conveniently and without discomfort for the subject during a long time. The signals transmitted and received by the first and second CW radars are single-frequency CW signals which are different from those by the UWB impulse radars in prior arts. Thanks to this feature, the system of the present invention has lower cost and better penetrating capability to measure PTT when an obstacle (e.g. cloth, bandage or hair) is present between the system and the skin. Moreover, a larger distance between the first and second positions can be set to reduce the calculation error of the pulse wave velocity.

### DESCRIPTION OF THE DRAWINGS

FIG. 1 is a diagram illustrating a conventional PTT measurement system.

FIG. 2 is a circuit diagram illustrating a non-contact PTT measurement system in accordance with a first embodiment of the present invention.

FIG. 3 is a diagram illustrating a wrist-worn smart device with the non-contact PTT measurement system in accordance with the first embodiment of the present invention.

FIG. 4 is a diagram illustrating a smart cloth with the non-contact PTT measurement system in accordance with the first embodiment of the present invention.

FIG. 5 represents measured waveforms of chest ECG and finger PPG by using the conventional PTT measurement system.

FIG. 6 represents measured waveforms of chest and wrist movements by using the wrist-worn smart device with the non-contact PTT measurement system of the present invention.

FIG. 7 represents measured waveforms of chest and wrist movements by using the smart cloth with the non-contact PTT measurement system of the present invention.

FIG. 8 shows a correlation between the PTTs measured by the present and conventional systems.

FIG. 9 is a circuit diagram illustrating a non-contact PTT measurement system in accordance with a second embodiment of the present invention.

FIG. 10 is a circuit diagram illustrating a non-contact PTT measurement system in accordance with a third embodiment of the present invention.

FIG. 11 is a circuit diagram illustrating a non-contact PTT measurement system in accordance with a fourth embodiment of the present invention.

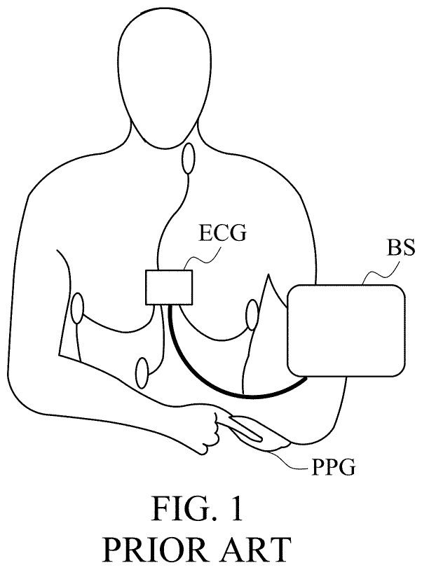

### DETAILED DESCRIPTION OF THE INVENTION

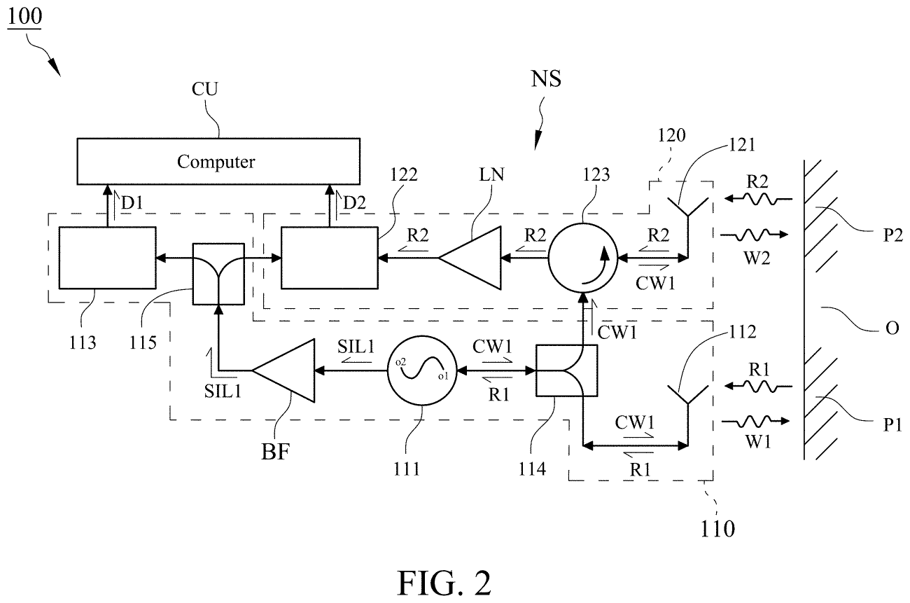

FIG. 2 is a circuit diagram of a non-contact pulse transit time (PTT) measurement system 100 in accordance with a first embodiment of the present invention. The non-contact PTT measurement system 100 includes a non-contact vital sign sensing device NS and a computer CU, and there are a first continuous-wave (CW) radar 110 and a second continuous-wave (CW) radar 120 in the non-contact vital sign sensing device NS.

With reference to FIG. 2, in the first embodiment, the first CW radar 110 is a self-injection-locked (SIL) radar and the second CW radar 120 is a direct-conversion radar. The first CW radar 110 includes a first oscillator 111, a first antenna 112, a first demodulator 113, a first power splitter 114 and a second power splitter 115. The first power splitter 114 and the second power splitter 115 are coupled to the first oscillator 111, the first antenna 112 is coupled to the first power splitter 114, and the first demodulator 113 is coupled to the second power splitter 115.

With reference to FIG. 2, the first oscillator 111 is configured to output a first continuous-wave (CW) signal CW1 and the first power splitter 114 is configured to receive and divide the first CW signal CW1 into two paths. The first CW signal CW1 of one path is delivered to the first antenna 112 and the first CW signal CW1 of the other path is delivered to the second CW radar 120. The first antenna 112 is configured to transmit the first CW signal CW1 as a first wireless signal W1 to a first position P1 on a subject O.

With reference to FIG. 2, the first wireless signal W1 transmitted to the first position P1 is reflected from the first position P1 as a first reflected signal R1. Based on the Doppler Effect, the first reflected signal R1 contains the Doppler phase shifts caused by the movement of the first position P1. The first reflected signal R1 from the first position P1 is received by the first antenna 112 and injected into the first oscillator 111 via the first power splitter 114 such that the first oscillator 111 enters a SIL state and outputs a first SIL signal SIL1. In the SIL state, the first SIL signal SIL1 from the first oscillator 111 produces a frequency variation in proportion to the Doppler phase shifts contained in the first reflected signal R1.

With reference to FIG. 2, the second power splitter 115 is configured to receive the first SIL signal SIL1 from the first oscillator 111 and divide the first SIL signal SIL1 into two paths. The first SIL signal SIL1 of one path is delivered to the first demodulator 113 and the first SIL signal SIL1 of the other path is delivered to the second CW radar 120. The first demodulator 113 is configured to receive and frequency-demodulate the first SIL signal SIL1 in order to obtain a first demodulated signal D1 for measuring the movement of the first position P1. Preferably, the second power splitter 115 is coupled to the first oscillator 111 via a buffer amplifier BF to prevent the oscillation frequency of the first oscillator 111 from varying due to impedance change in the second power splitter 115.

With reference to FIG. 2, the second CW radar 120 includes a second antenna 121, a second demodulator 122 and a circulator 123. The circulator 123 is coupled to the first power splitter 114 of the first CW radar 110, the second antenna 121 and the second demodulator 122. The first CW signal CW1 of the other path front the first power splitter 114 is received and delivered to the second antenna 121 by the circulator 123. The second antenna 121 is configured to transmit the first CW signal CW1 as a second wireless signal W2 to a second position P2 on the subject O.

With reference to FIG. 2, the second wireless signal W2 transmitted to the second position P2 is reflected from the second position P2 as a second reflected signal R2. For the same reason, the second reflected signal R2 also contains the Doppler phase shifts resulting from the movement of the second position P2. The second reflected signal R2 is received by the second antenna 121 and then sent to the circulator 123. The circulator 123 is configured to deliver the second reflected signal R2 to the second demodulator 122. The circulator 123 is designed not to deliver the second reflected signal R2 to the first power splitter 114, so the second reflected signal R2 will not enter the first oscillator 111 to change the oscillation frequency of the first oscillator 111.

With reference to FIG. 2, the second demodulator 122 is coupled to the second antenna 121 via the circulator 123 and configured to receive the second reflected signal R2 and the first SIL signal SIL1 from the other path of the second power splitter 115 of the first CW radar 110. The second demodulator 122 uses the first SIL signal SIL1 as a reference signal to phase-demodulate the second reflected signal R2 so as to obtain a second demodulated signal D2 for measuring the movement of the second position P2. Preferably, the second demodulator 122 is coupled to the circulator 123 via a low-noise amplifier LN to amplify the second reflected signal R2 and thus improve the signal-to-noise ratio of the second demodulated signal D2.

With reference to FIG. 2, the computer CU is coupled to the first CW radar 110 and the second CW radar 120 and configured to receive the first demodulated signal D1 and the second demodulated signal D2 from the first demodulator 113 and the second demodulator 122, respectively, for use in extracting and providing the PTT between the first position P1 and the second position P2. Then, the pulse wave velocity can be calculated accordingly to estimate the blood pressure (BP).

With reference to FIG. 2, since the second CW radar 120 does not have its own oscillator, the power consumption of the second CW radar 120 can be reduced and the interference between the first CW radar 110 and the second CW radar 120 in the non-contact vital sign sensing device NS can be avoided.

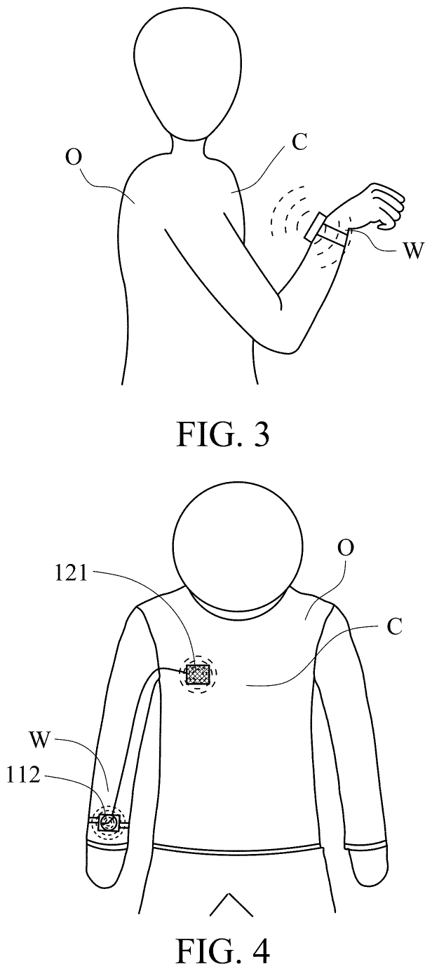

With reference to FIG. 3, the first position P1 and the second position P2 in this embodiment are on a wrist W and a chest C of the subject O, respectively. The movement waveform at the first position P1 represents the instantaneous vibration caused by the pulse waves passing the first position P1 on the wrist W and the movement waveform at the second position P2 represents the instantaneous vibration caused by the pulse waves passing the position P2 on the chest C. The PTT is the travel time of the pulse wave between the first position P1 and the second position P2.

With reference to FIG. 3, the non-contact PTT measurement system 100 of the present invention may be installed in a smart device (e.g. smart watch or smart wristband) word on the wrist W of the subject O. The first antenna 112 and the second antenna 121 used in the wrist-worn smart device can make no contact with the skin and their beams are directed to the wrist W and chest C of the subject O, respectively, for PTT measurement. Alternatively, the non-contact PTT measurement system 100 may be a smart cloth as shown in FIG. 4, and the first antenna 112 and the second antenna 121 are embedded in the smart cloth near the wrist W and the chest C of the subject O, respectively, without need to contact to the skin. The smart cloth can keep the antenna beams directed to the wrist W and the chest C more easily to provide more stable PTT measurement.

In other embodiments, the first position P1 and the second position P2 may be two different positions on the same region of the subject O. Additionally, the system of the present invention utilizes two single-frequency CW radars, achieving higher penetration through the obstacles than UWB systems because of higher transmit power. Therefore, there is no need to place the antennas close to the skin when the system of the prevent invention carries out the detection of pulse wave signals. For this reason, the PTT between two far away positions on the subject O is measurable. Preferably, the distance between the first position P1 and the second position P2 is set more than 10 cm to reduce the influence of the error in the PTT on the calculation accuracy of the pulse wave velocity.

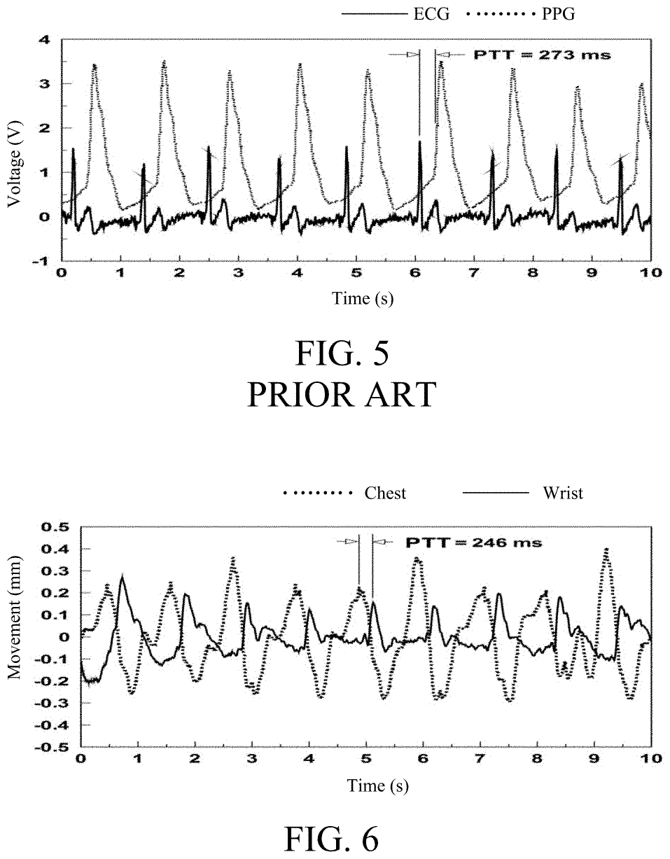

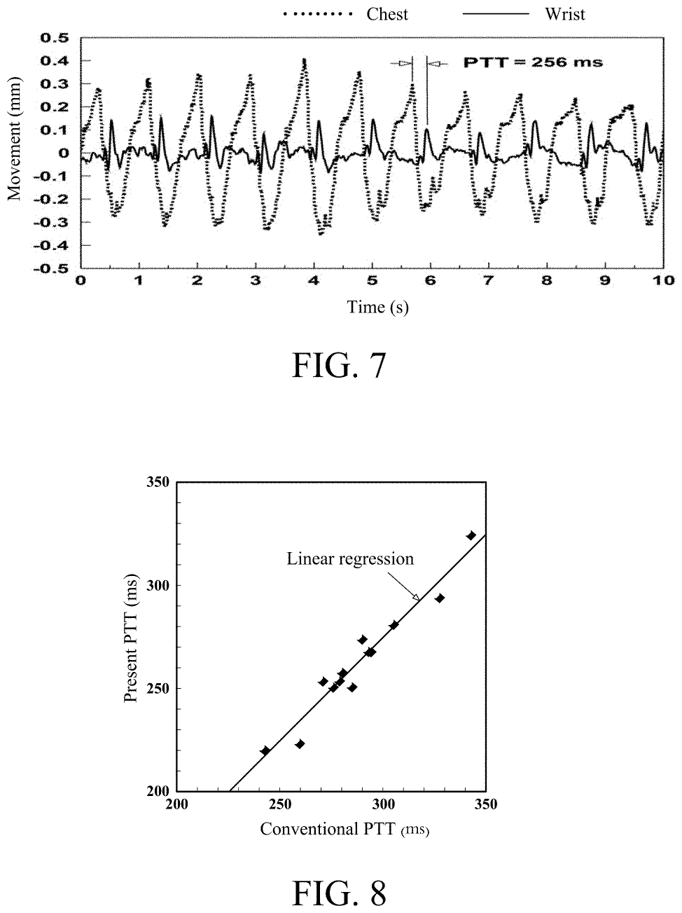

FIG. 5 shows the measured chest-ECG and finger-PPG signals of a 28 year old subject using a conventional PTT measurement system. The average PTT estimated by the time difference between the chest-ECG and finger-PPG signals is 273 ms. FIG. 6 and FIG. 7 represent the measured movement signals at the wrist and chest of the same subject by using the wrist-worn smart device and the smart cloth, respectively, with the first embodiment. According to the time difference between the chest-movement and wrist-movement signals, the average PTT estimated from FIG. 6 and FIG. 7 is 246 and 256 ms, respectively, and there is a difference of 10 ms because the antennas in the wrist-worn smart device and the smart cloth are directed to slightly different positions on the subject. Moreover, the average PTT estimated from FIG. 6 and FIG. 7 is less by 27 and 17 ms, respectively, than that estimated from FIG. 5. This is because the wrist-worn smart device and the smart cloth with the first embodiment are designed to measure the PTT from chest to wrist, differing from the conventional system that measures the PTT from chest to finger. Therefore, their difference in the PTT is attributed to the travel time of the pulse wave from wrist to finger. This comparison supports that the non-contact PTT measurement system 100 of the present invention can measure the PTT between two far away positions on the subject's body accurately.

With reference to FIG. 8, it shows the comparison between the conventional and present PTT measurements of 13 subjects aged from 22 to 28 years. The PTT measured by the present system ranges from 220 to 320 ms. The regression line in FIG. 8 has a root-mean-square error of 6.1 ms, revealing that the PTT measurements using the conventional and present systems correlate well with each other.

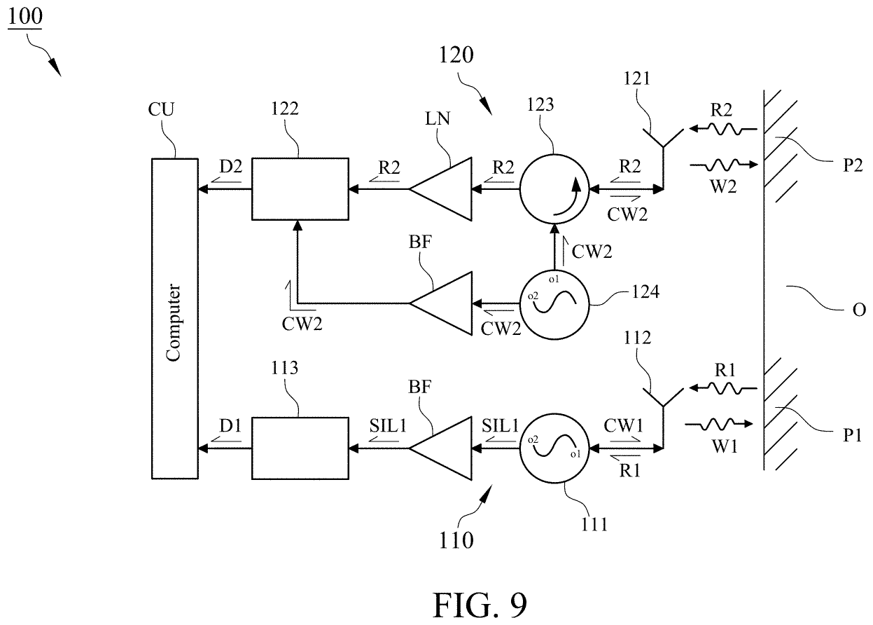

With reference to FIG. 9, a non-contact PTT measurement system 100 of a second embodiment includes a first CW radar 110, a second CW radar 120 and a computer CU. The first CW radar 110 is a SIL radar and the second CW radar 120 is a direct-conversion radar having a second oscillator 124, a circulator 123, a second antenna 121 and a second demodulator 122. Differing from the first embodiment, the second CW radar 120 of the second embodiment has its own oscillator to provide the reference signal.

With reference to FIG. 9, the circulator 123 is coupled to the second oscillator 124 and the second antenna 121, and the second demodulator 122 is coupled to the circulator 123 and the second oscillator 124. The second oscillator 124 is configured to output a second continuous-wave signal CW2, the circulator 123 is configured to deliver the second CW signal CW2 to the second antenna 121, and the second antenna 121 is configured to transmit the second CW signal CW2 as a second wireless signal W2 to a second position P2 on a subject O. The second wireless signal W2 is reflected from the second position P2 as a second reflected signal R2 that contains the Doppler phase shifts caused by the movement of the second position P2.

With reference to FIG. 9, the second reflected signal R2 is received by the second antenna 121 and then delivered to the circulator 123, and the second demodulator 122 is configured to receive the second reflected signal R2 from the circulator 123 and the second CW signal CW2 from the second oscillator 124. The second demodulator 122 uses the second CW signal CW2 as a reference signal to phase-demodulate the second reflected signal R2 such that a second demodulated signal D2 is obtained for measuring the movement of the second position P2. Preferably, a low-noise amplifier LN is coupled to the second demodulator 122 and the circulator 123 to amplify the second reflected signal R2 and thus improve the signal-to-noise ratio of the second demodulated signal D2. Moreover, a buffer amplifier BF is coupled to the second demodulator 122 and the second oscillator 124 to prevent the oscillation frequency of the second oscillator 124 from varying due to impedance change in the second demodulator 122.

With reference to FIG. 9, the first CW radar 110 of the second embodiment doesn't require the first power splitter 114 and the second power splitter 115 used in the first embodiment because the second CW radar 120 of the second embodiment has its own oscillator to provide the reference signal. In the second embodiment, the first CW radar 110 includes a first oscillator 111, a first antenna 112 and a first demodulator 113, and the first antenna 112 and the first demodulator 113 are coupled to the first oscillator 111. The first oscillator 111 is configured to output a first CW signal CW1, the first antenna 112 is configured to transmit the first CW signal CW1 as a first wireless signal W1 to a first position P1 on the subject O. The first wireless signal W1 transmitted to the first position P1 is reflected as a first reflected signal R1 that contains the Doppler phase shifts caused by the movement of the first position P1. The first antenna 112 receives the first reflected signal R1 from the first position P1 and injects the first reflected signal R1 into the first oscillator 111 such that the first oscillator 111 enters a SIL state and outputs a first SIL signal SIL1. In the SIL state, the first SIL signal SIL1 from the first oscillator 111 produces a frequency variation in proportion to the Doppler phase shifts contained in the first reflected signal R1. The first demodulator 113 is configured to receive and frequency-demodulate the first SIL signal SIL1 for measuring the movement of the first position P1. Preferably, a buffer amplifier BF is coupled to the first demodulator 113 and the first oscillator 111 to prevent the oscillation frequency of the first oscillator 111 from varying due to impedance change in the first demodulator 113.

With reference to FIG. 9, the computer CU is coupled to the first demodulator 113 and the second demodulator 122 to receive the first demodulated signal D1 and the second demodulated signal D2. In the second embodiment, the computer CU is also configured to extract the PTT between the first position P1 and the second position P2 from the first demodulated signal D1 and the second demodulated signal D2 and then calculate the pulse wave velocity accordingly to estimate the BP.

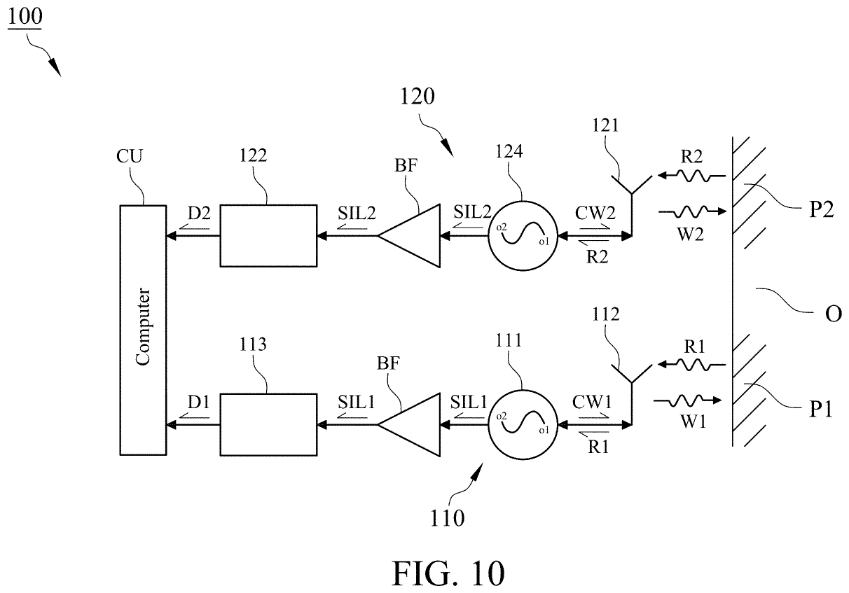

FIG. 10 shows a circuit diagram of a non-contact PTT measurement system 100 of a third embodiment. The non-contact PTT measurement system 100 includes a first CW radar 110, a second CW radar 120 and a computer CU, and the first CW radar 110 and the second CW radar 120 are both SIL radars. The second CW radar 120 includes a second oscillator 124, a second antenna 121 and a second demodulator 122, and the second antenna 121 and the second demodulator 122 are coupled to the second oscillator 124. A second CW signal CW2 from the second oscillator 124 is transmitted via the second antenna 121 as a second wireless signal W2 to a second position P2 of a subject O. The second wireless signal W2 is reflected from the second position P2 as a second reflected signal R2 that contains the Doppler phase shifts caused by the movement of the second position P2. The second antenna 121 receives the second reflected signal R2 from the second position P2 and injects the second reflected signal R2 into the second oscillator 124 to make the second oscillator 124 enter a SIL state and output a second SIL signal SIL2. In the SIL state, the second SIL signal SIL2 from the second oscillator 124 produces a frequency variation in proportion to the Doppler phase shifts contained in the second reflected signal R2. The second demodulator 122 is configured to receive and frequency-demodulate the second SIL signal SIL2 to produce a second demodulated signal D2 for measuring the movement of the second position P2. Moreover, the second demodulator 122 is preferably coupled to the second oscillator 124 via a buffer amplifier BF to prevent the oscillation frequency of the second oscillator 124 from varying due to impedance change in the second demodulator 122.

With reference to FIG. 10, the first CW radar 110 includes a first oscillator 111, a first antenna 112 and a first demodulator 113, and the first antenna 112 and the first demodulator 113 are coupled to the first oscillator 111. The first antenna 112 is configured to transmit the first CW signal CW1 generated by the first oscillator 111 to a first position P1 on the subject O as a first wireless signal W1. The first wireless signal W1 is reflected from the first position P1 on the subject O as a first reflected signal R1 that contains the Doppler phase shifts caused by the movement of the first position P1. The first reflected signal R1 received by the first antenna 112 is injected into the first oscillator 111 to make the first oscillator 111 enter a SIL state and output a first SIL signal SIL1. In the SIL state, the first SIL signal SIL1 from the first oscillator 111 produces a frequency variation in proportion to the Doppler phase shifts contained in the first reflected signal R1. The first demodulator 113 is configured to receive and frequency-demodulate the first SIL signal SIL1 to produce a first demodulated signal D1 for measuring the movement of the first position P1. A buffer amplifier BF is preferably coupled to the first oscillator 111 and the first demodulator 113 to prevent the oscillation frequency of the first oscillator 111 from varying due to impedance change in the first demodulator 113.

With reference to FIG. 10, the computer CU is coupled to the first demodulator 113 and the second demodulator 122 for receiving the first demodulated signal D1 and the second demodulated signal D2. The computer CU of the third embodiment also serves to extract the PTT between the first position P1 and the second position P2 from the first demodulated signal D1 and the second demodulated signal D2 for calculating the pulse wave velocity and subsequently estimating the BP.

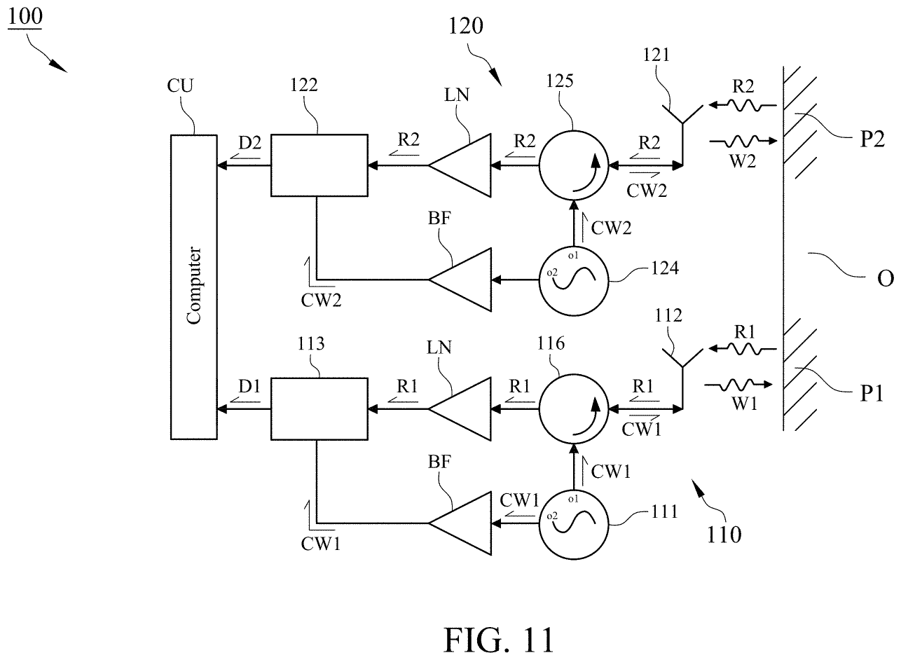

With reference to FIG. 11, it is a circuit diagram of a non-contact PTT measurement system 100 of a fourth embodiment, the system 100 includes a first CW radar 110, a second CW radar 120 and a computer CU. In the fourth embodiment, both of the first CW radar 110 and the second CW radar 120 are direct-conversion radars. There are a first oscillator 111, a first circulator 116, a first antenna 112 and a first demodulator 113 in the first CW radar 110. The first circulator 116 is coupled to the first oscillator 111 and the first antenna 112, and the first demodulator 113 is coupled to the first circulator 116 and the first oscillator 114. The first oscillator 111 is configured to output a first CW signal CW1, the first circulator 116 is configured to receive and deliver the first CW signal CW1 to the first antenna 112, then the first antenna 112 is configured to transmit the first CW signal CW1 as a first wireless signal W1 to a first position P1 of a subject O. The first wireless signal W1 transmitted to the first position P1 is reflected as a first reflected signal from the first position P1. Based on the Doppler Effect, the first reflected signal R1 contains the Doppler phase shifts caused by the movement of the first position P1. The first antenna 112 is configured to receive and deliver the first reflected signal R1 to the first circulator 116, and the first demodulator 113 is configured to receive the first reflected signal R1 from the first circulator 116 and also receive the first CW signal CW1 from the first oscillator 111. The first demodulator 113 is configured to phase-demodulate the first reflected signal R1 by using the first CW signal CW1 as a reference signal so as to obtain a first demodulated signal D1 for measuring the movement of the first position P1. Preferably, the first demodulator 113 is coupled to the first circulator 116 via a low-noise amplifier LN to amplify the first reflected signal R1 and thus improve the signal-to-noise ratio of the first demodulated signal D1. Meanwhile, the first CW signal CSW1 from the first oscillator 111 is delivered to the first demodulator 113 via a buffer amplifier BF to prevent the oscillation frequency of the first oscillator 111 from varying due to impedance change in the first demodulator 113.

With reference to FIG. 11, the second CW radar 120 includes a second oscillator 124, a second circulator 125, a second antenna 121 and a second demodulator 122. The second circulator 125 is coupled to the second oscillator 124 and the second antenna 121, the second demodulator 122 is coupled to the second circulator 125 and the second oscillator 124. The second circulator 125 is configured to receive a second CW signal CW2 from the second oscillator 124 and deliver the second CW signal CW2 to the second antenna 121. The second antenna 121 is configured to transmit the second CW signal CW2 as a second wireless signal W2 to a second position P2 on the subject O and receive a second reflected signal R2 reflected from the second position P2. Notably, the second reflected signal R2 contains the Doppler phase shifts caused by the movement of the second position P2. The second antenna 121 receives and delivers the second reflected signal R2 to the second circulator 125, the second demodulator 122 is configured to receive the second reflected signal R2 from the second circulator 125 and receive the second CW signal CW2 from the second oscillator 124 to phase-demodulate the second reflected signal R2 by using the second CW signal CW2 as a reference signal and produce a second demodulated signal D2 from which the movement of the second position P2 can be measured. Preferably, the second demodulator 122 is coupled to the second circulator 125 via a low-noise amplifier LN to amplify the second reflected signal R2 and thus improve the signal-to-noise ratio of the second demodulated signal D2. Furthermore, the second CW signal CW2 is delivered from the second oscillator 124 to the second demodulator 122 via a buffer amplifier BF to prevent the oscillation frequency of the second oscillator 124 from varying due to impedance change in the second demodulator 122.

With reference to FIG. 11, the computer CU is coupled to the first demodulator 113 and the second demodulator 122 so as to receive the first demodulated signal D1 and the second demodulated signal D2. The computer CU can utilize the first demodulated signal D1 and the second demodulated signal D2 to extract the PTT between the first position P1 and the second position P2 and then calculate the pulse wave velocity from the PTT to estimate the BP.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A non-contact pulse transit time measurement system comprising:
a non-contact vital sign sensing device including:
a first continuous-wave (CW) radar configured to transmit a first wireless signal to a first position on a subject, receive a first reflected signal reflected from the first position, and perform demodulation according to the first reflected signal to obtain a first demodulated signal; and
a second continuous-wave (CW) radar configured to transmit a second wireless signal to a second position on the subject, receive a second reflected signal reflected from the second position, and perform demodulation according to the second reflected signal to obtain a second demodulated signal; and
a computer coupled to the first and second CW radars of the non-contact vital sign sensing device for receiving the first and second demodulated signals from the first and second CW radars and configured to extract a pulse transit time from the first and second demodulated signals.

2. The non-contact pulse transit time measurement system in accordance with claim 1, wherein the first CW radar includes a first oscillator, a first antenna and a first demodulator, the first oscillator is configured to generate a first continuous-wave (CW) signal, the first antenna is coupled to the first oscillator and configured to transmit the first CW signal as the first wireless signal to the first position on the subject, the first reflected signal reflected from the first position is received by the first antenna and injected into the first oscillator such that the first oscillator enters a self-injection-locked (SIL) state and outputs a first SIL signal, the first demodulator is coupled to the first oscillator and configured to receive and frequency-demodulate the first SIL signal so as to obtain the first demodulated signal.

3. The non-contact pulse transit time measurement system in accordance with claim 2, wherein the second CW radar includes a second antenna and a second demodulator, the second antenna is coupled to the first oscillator and configured to receive the first CW signal, transmit the first CW signal as the second wireless signal to the second position on the subject and receive the second reflected signal reflected from the second position, the second demodulator is coupled to the second antenna and configured to receive and demodulate the second reflected signal.

4. The non-contact pulse transit time measurement system in accordance with claim 3, wherein the first CW radar further includes a first power splitter and the second CW radar further includes a circulator, the circulator is coupled to the first power splitter, the second antenna and the second demodulator, the first power splitter is coupled to the first oscillator and configured to divide the first CW signal into two paths, wherein the first CW signal of one path is delivered to the first antenna and the first CW signal of the other path is delivered to the circulator, the circulator is configured to deliver the first CW signal to the second antenna and deliver the second reflected signal received by the second antenna to the second demodulator.

5. The non-contact pulse transit time measurement system in accordance with claim 4, wherein the first CW radar further includes a second power splitter that is coupled to the first oscillator, the first demodulator and the second demodulator, the second power splitter is configured to divide the first SIL signal generated by the first oscillator into two paths, wherein the first SIL signal of one path is delivered to the first demodulator and the first SIL signal of the other path is delivered to the second demodulator, the second demodulator is configured to phase-demodulate the second reflected signal by using the first SIL signal as a reference signal to obtain the second demodulated signal.

6. The non-contact pulse transit time measurement system in accordance with claim 2, wherein the second CW radar includes a second oscillator, a circulator, a second antenna and a second demodulator, the second oscillator is configured to generate a second continuous-wave (CW) signal, the circulator is coupled to the second oscillator, the second antenna and the second demodulator and configured to deliver the second CW signal generated by the second oscillator to the second antenna, the second antenna is configured to transmit the second CW signal as the second wireless signal to the second position on the subject, receive the second reflected signal reflected from the second position and deliver the second reflected signal to the circulator, the circulator is configured to deliver the second reflected signal to the second demodulator, the second demodulator is coupled to the second oscillator for receiving the second CW signal and configured to phase-demodulate the second reflected signal by using the second CW signal as a reference signal to obtain the second demodulated signal.

7. The non-contact pulse transit time measurement system in accordance with claim 2, wherein the second CW radar includes a second oscillator, a second antenna and a second demodulator, the second oscillator is configured to generate a second continuous-wave (CW) signal, the second antenna is coupled to the second oscillator and configured to transmit the second CW signal as the second wireless signal to the second position on the subject, the second reflected signal reflected from the second position is received by the second antenna and injected into the second oscillator such that the second oscillator enters a SIL state and outputs a second SIL signal, the second demodulator is coupled to the second oscillator for receiving the second SIL signal and configured to frequency-demodulate the second SIL signal to obtain the second demodulated signal.

8. The non-contact pulse transit time measurement system in accordance with claim 1, wherein the first CW radar includes a first oscillator, a first circulator, a first antenna and a first demodulator, the first oscillator is configured to generate a first continuous-wave (CW) signal, the first circulator is coupled to the first oscillator, the first antenna and the first demodulator and configured to deliver the first CW signal to the first antenna, the first antenna is configured to transmit the first CW signal as the first wireless signal to the first position on the subject, receive the first reflected signal reflected from the first position and deliver the first reflected signal to the first circulator, the first circulator is configured to deliver the first reflected signal to the first demodulator, the first demodulator is coupled to the first oscillator for receiving the first CW signal and configured to phase-demodulate the first reflected signal by using the first CW signal as a reference signal to obtain the first demodulated signal.

9. The non-contact pulse transit time measurement system in accordance with claim 8, wherein the second CW radar includes a second oscillator, a second circulator, a second antenna and a second demodulator, the second oscillator is configured to generate a second continuous-wave (CW) signal, the second circulator is coupled to the second oscillator, the second antenna and the second demodulator and configured to deliver the second CW signal to the second antenna, the second antenna is configured to transmit the second CW signal as the second wireless signal to the second position on the subject, receive the second reflected signal reflected from the second position and deliver the second reflected signal to the second circulator, the second circulator is configured to deliver the second reflected signal to the second demodulator, the second demodulator is coupled to the second oscillator for receiving the second CW signal and configured to phase-demodulate the second reflected signal by using the second CW signal as a reference signal to obtain the second demodulated signal.

10. The non-contact pulse transit time measurement system in accordance with claim 1, wherein a distance between the first and second positions on the subject is larger than 10 cm.

11. The non-contact pulse transit time measurement system in accordance with claim 3, wherein the non-contact pulse transit time measurement system is integrated in a wearable device, and beams of the first and second antennas are directed toward the first and second positions on the subject respectively.

12. The non-contact pulse transit time measurement system in accordance with claim 6, wherein the non-contact pulse transit time measurement system is integrated in a wearable device, and beams of the first and second antennas are directed toward the first and second positions on the subject respectively.

13. The non-contact pulse transit time measurement system in accordance with claim 7, wherein the non-contact pulse transit time measurement system is integrated in a wearable device, and beams of the first and second antennas are directed toward the first and second positions on the subject respectively.

14. The non-contact pulse transit time measurement system in accordance with claim 9, wherein the non-contact pulse transit time measurement system is integrated in a wearable device, and beams of the first and second antennas are directed toward the first and second positions on the subject respectively.

15. A non-contact vital sign sensing device comprising:
an oscillator configured to generate a first continuous-wave (CW) signal;
a first power splitter coupled to the oscillator and configured to divide the first CW signal into two paths;
a first antenna coupled to the first power splitter for receiving the first CW signal of one path and configured to transmit the first CW signal as a first wireless signal to a first position on a subject and receive a first reflected signal reflected from the first position, wherein the first reflected signal is injected into the oscillator via the first power splitter such that the oscillator enters a SIL state and outputs a first SIL signal;
a circulator coupled to the first power splitter for receiving the first CW signal of the other path;
a second antenna coupled to the circulator, wherein the circulator is configured to deliver the first CW signal to the second antenna and the second antenna is configured to transmit the first CW signal as a second wireless signal to a second position on the subject, receive a second reflected signal reflected from the second position and deliver the second reflected signal to the circulator;
a second power splitter coupled to the oscillator and configured to receive and divide the first SIL signal into two paths;
a first demodulator coupled to the second power splitter for receiving the first SIL signal of one path and configured to frequency-demodulate the first SIL signal to obtain a first demodulated signal; and
a second demodulator coupled to the circulator and the second power splitter and configured to receive the second reflected signal from the circulator, receive the first SIL signal of the other path from the second power splitter and phase-demodulate the second reflected signal by using the first SIL signal as a reference signal to obtain a second demodulated signal.

16. The non-contact vital sign sensing device in accordance with claim further comprising a buffer amplifier, wherein the buffer amplifier is coupled to the oscillator, and the second power splitter is coupled to the oscillator via the buffer amplifier.

17. The non-contact vital sign sensing device in accordance with claim 15 further comprising a low-noise amplifier, wherein the low-noise amplifier is coupled to the circulator, and the second demodulator is coupled to the circulator via the low-noise amplifier.

18. The non-contact vital sign sensing device in accordance with claim 15, wherein the first and second demodulated signals are provided to analyze vital signs of the subject, and the vital signs include respiration, heartbeat and blood pressure.

19. The non-contact vital sign sensing device in accordance with claim 15, wherein there is only a single oscillator in the non-contact vital sign sensing device.
